(19) United States
(12) Patent Application Publication
(10) Pub. No.: US 2026/0174347 A1
(43) Pub. Date: Jun. 25, 2026

(54) OPTICAL COATINGS FOR GLASS SHEETS IN WEARABLE DEVICES

(71) Applicant: Oura Health Oy, Oulu (FI)

(72) Inventors: Jukka Tapani Mäkinen, Oulu (FI); Mika Petteri Kangas, Oulu (FI); Teemu Juhani Haverinen, Oulu (FI); Heikki Huttunen, Oulu (FI)

(21) Appl. No.: 19/423,971

(22) Filed: Dec. 17, 2025

Related U.S. Application Data
(60) Provisional application No. 63/736,485, filed on Dec. 19, 2024.

(51) Int. Cl. A61B 5/024; A61B 5/00; A61B 5/1455; C09D 1/00; G02B 6/42

(52) U.S. Cl. CPC A61B 5/02427; A61B 5/02405; A61B 5/14552; A61B 5/4815; A61B 5/6802; C09D 1/00; G02B 6/4214; G02B 6/4298; A61B 2562/0219; A61B 2562/0233; A61B 2562/166

(57) ABSTRACT

Methods, systems, and devices for optical coatings for glass sheets in wearable devices are described. The wearable device ring may include a housing and one or more light sources and one or more detectors at least partially disposed within the housing. The wearable ring device may include a layer of glass at least partially coupled to the housing and positioned to receive at least a first portion of light emitted from the one or more light sources. In some cases, the wearable ring device may include one or more reflective surfaces coupled to the layer of glass and positioned to reflect at least a portion of the first portion of light.

### CROSS REFERENCE

The present application for patent claims priority to U.S. Provisional Patent Application No. 63/736,485 by Makinen et al., entitled “OPTICAL COATINGS FOR GLASS SHEETS IN WEARABLE DEVICES” filed Dec. 19, 2024. Application No. 63/736,485 is herein incorporated by reference in its entirety.

### FIELD OF TECHNOLOGY

The following relates to wearable devices and data processing, including optical coatings for glass sheets in wearable devices.

### BACKGROUND

Some wearable devices may be configured to collect data from users, including temperature data, heart rate data, and the like. However, light that travels directly between a light source and a detector of the wearable device without traveling through a user's skin may result in inaccurate measurements.

### BRIEF DESCRIPTION OF THE DRAWINGS

FIG. 1 shows an example of wearable device diagrams that supports optical coatings for glass sheets in wearable devices in accordance with aspects of the present disclosure.

FIG. 2 shows an example of wearable device diagrams that supports optical coatings for glass sheets in wearable devices in accordance with aspects of the present disclosure.

FIG. 3 shows an example of wearable device diagrams with a light guide that supports optical coatings for glass sheets in wearable devices in accordance with aspects of the present disclosure.

FIG. 4 shows an example of wearable device diagrams with phosphor material that supports optical coatings for glass sheets in wearable devices in accordance with aspects of the present disclosure.

FIG. 5 shows an example of wearable device diagrams with phosphor materials that supports optical coatings for glass sheets in wearable devices in accordance with aspects of the present disclosure.

FIG. 6 shows an example of a wearable device diagram with one or more metal wires that supports optical coatings for glass sheets in wearable devices in accordance with aspects of the present disclosure.

FIGS. 7 and 8 illustrate examples of systems that support optical coatings for glass sheets in wearable devices in accordance with aspects of the present disclosure.

### DETAILED DESCRIPTION

Some wearable devices may be configured to collect data from users associated with movement and other activities. For example, some wearable devices may be configured to continuously acquire physiological data associated with a user including temperature data, heart rate data, and the like. As such, some wearable devices may be configured to house one or more physiological sensors configured to acquire physiological data from a user. In some cases, a wearable device may include a flexible printed circuit board (PCB) including electrical circuitry for the one or more physiological sensors. The wearable device may include one or more light sources (e.g., light emitting diodes (LEDs), laser diodes (LDs), vertical cavity surface-emitting lasers (VCSELs), and the like other types of light sources) positioned to direct light into a tissue surface of the user and one or more detectors (e.g., photodetectors) positioned to receive the light that passes at least partially through the tissue surface.

A user's movement, or the movement of the wearable with respect to the user's body (e.g., rotation, vibration), or the fit of the wearable on the user may detrimentally affect the ability of the wearable device to efficiently and accurately acquire physiological data and may increase an amount of noise in the signal. This issue with wearable devices may result in inaccurate physiological data readings, which may lead to a distorted picture of the user's overall health, as well as increased power consumption and decreased battery life. As such, conventional techniques for obtaining optical measurements may be improved.

Accordingly, to facilitate improved health monitoring, aspects of the present disclosure are directed to optical coatings for glass sheets in wearable devices. For example, the wearable device may include a layer of glass disposed on the surface of a housing. The layer of glass may be generally disposed over one or more optical components such as one or more light sources and one or more detectors. The layer of glass may include one or more reflective surfaces. For example, the one or more reflective surfaces may be positioned to reflect at least a portion of the light emitted from the light source. The one or more reflective surfaces are configured to modify a set of characteristics of a light emission pattern of the light sources towards the detectors.

In such cases, the one or more reflective surfaces may help direct or focus the light emission pattern of the one or more light sources to be directionally towards the field of view of the detectors, thereby decreasing an amount of noise in the signal and increasing the efficiency and accuracy of the signal. By implementing the one or more reflective surfaces on a surface of the layer of glass within the housing of the wearable device, techniques described herein may lead to more accurate physiological data measurements.

Aspects of the disclosure are initially described in the context of systems supporting physiological data collection from users via wearable devices. Additional aspects of the disclosure are described in the context of wearable device diagrams. Aspects of the disclosure are further illustrated by and described with reference to apparatus diagrams, system diagrams, and flowcharts that relate to optical coatings for glass sheets in wearable devices.

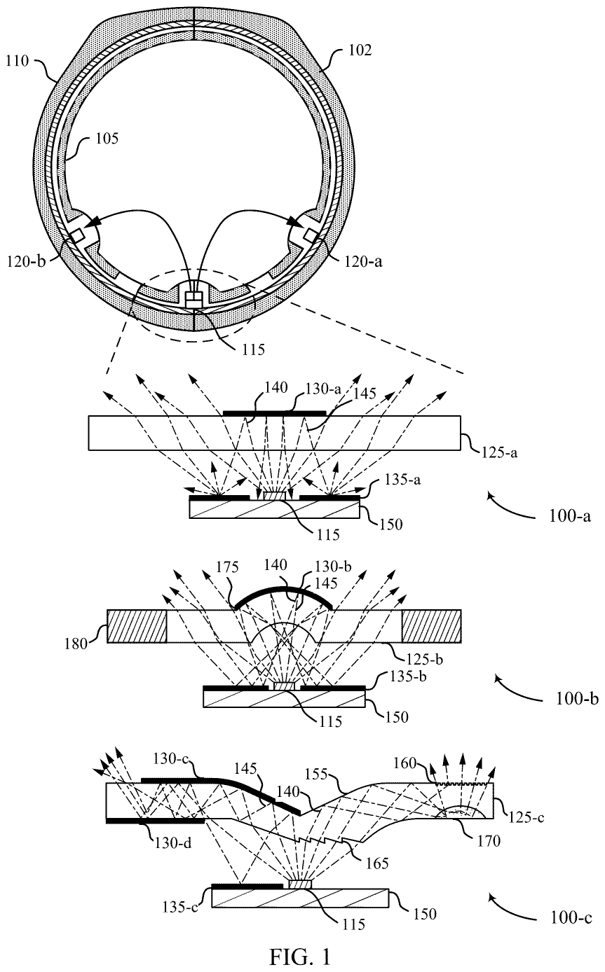

FIG. 1 shows an example of wearable device diagrams 100 that supports optical coatings for glass sheets in wearable devices in accordance with aspects of the present disclosure. Wearable device diagram 100 may illustrate examples of wearable device 102. Although the wearable device 102 is illustrated as a ring in FIG. 1, aspects and components of the wearable device 102 illustrated in FIG. 1 may be implemented in any type of wearable device (e.g., a watch, a bracelet, a necklace, and the like).

The wearable device 102 in wearable device diagrams 100 may include a housing that includes an inner housing 105 and an outer housing 110. The wearable device 102 may include a flexible printed circuit board (PCB) 150. The flexible PCB 150 may include one or more light sources 115 and detectors 120. The light sources 115 may be an example of light emitting diode (LED) lights that may be a blue LED light, a yellow LED light, a green LED light, a red light, an IR light, or some other color LED light. In some cases, the light sources 115 may be an example of a laser diode (LD) or a vertical-cavity surface-emitting laser (VCSEL).

The wearable device 102 may include light source 115, which may emit light that is then received by detector 120-a and/or detector 120-b. In this regard, the light source 115 may support one or more optical paths through the tissue for physiological data measurements. For instance, the light source 115 may support an optical path between the light source 115 and the detector 120-a and another optical path between the light source 115 and the detector 120-b. The wearable device 102 may include any number of light sources, detectors, and respective optical paths for physiological data measurements. In some cases, the light source 115 may be a red and infrared LED, which may emit light that is scattered and absorbed by the tissue of a user of the wearable device 102.

The detectors 120-a and 120-b may be configured to measure light from the respective light sources 115 which is reflected by the tissue and/or transmitted through the tissue (e.g., reflective and/or transmissive measurements). In such cases, the light may be used for physiological data measurements associated with the user.

In some systems, wearable device photoplethysmogram (PPG) sensors (e.g., light sources 115 and detectors 120) may be fitted into very small spaces within the wearable device 102. The light sources 115 and detectors 120 may be required to operate with high optical efficiency due to the limited power supply (e.g., a battery within the wearable device 102). However, the light sources 115 and detectors 120 may be sensitive to contact, acceleration, contaminants, and the like. For example, the wearable device 102 may be subjected to a force or an acceleration, causing an air gap between the surface of the tissue and the light sources 115 and the detectors 120 at the wearable device 102. The air gap between the tissue and the light sources 115 and the detectors 120 may disturb the optical paths, as light may be coupled to the tissue through two interfaces (e.g., the interface between the light sources 115 and the detectors 120 and the air and the interface between the air and the tissue). Additionally or alternatively, liquid or other contaminants may be trapped between the tissue and the light sources 115 and the detectors 120. The contaminants may dampen or absorb the optical signals. Further, the difference between refractive indexes and contaminant layer absorption spectra may determine how different signal paths/channels may be affected (e.g., causing increased variability in signal strength).

The inner housing 105 may include a layer of glass 125. In such cases, the layer of glass 125 (e.g., a thin glass sheet) may be implemented to protect the light sources 115 and the detectors 120 against the environment. The optical functioning of the light sources 115 and the detectors 120 may be enhanced with the different kinds of coatings on the layer of glass 125 without a large increase in sensor structure thickness (e.g., overall thickness of the light sources 115 and the detectors 120 and thereby the thickness of the wearable device 102). Changing the shape and/or coating of the layer of glass 125 may be used for changing an emission pattern of the light source 115 and/or a field of view of the detectors 120.

The layer of glass 125 may include one or more dome structures positioned over the one or more light sources 115, over the one or more detectors 120, or both. For example, the wearable device 102 may include dome structures over the light source 115, the detector 120-a, and the detector 120-b to improve contact with the tissue. In some other cases, rather than a dome structure as pictured, the layer of glass 125 may be flat over the light sources 115 or detectors 120. In other examples, the layer of glass 125 may be curved over the light sources 115 or detectors 120 to follow the general curvature of the inner housing 105. The wearable device 102 may use the light propagation from the light sources 115 to the detectors 120 through the tissue and along the one or more optical paths for physiological measurements, such as PPG and SpO2 measurements. That is, the wearable device 102 may use the light from the light source 115, which may include red and infrared wavelengths, to measure SpO2, among other physiological measurements.

The layer of glass 125 may include one or more reflective surfaces 130. The reflective surfaces 130 may be formed from one or more coatings applied to the layer of glass 125. Additionally or alternatively, the reflective surfaces 130 may be formed from one or more reflective components that are adhered to or embedded within the layer of glass 125. By using different kinds of reflective surfaces 130 on top of or embedded within the layer of glass 125, and by positioning them with respect to the light sources 115, a light emission pattern of the light sources 115 may be modified. The reflective surfaces 130 may be configured to alter the light pattern of the light emitted from the light sources 115 by reflecting light, focusing light, redirecting light, or any combination thereof.

Wearable device diagram 100-a depicts an example where the layer of glass 125-a is planar with respect to the flexible PCB 150. The layer of glass 125-a may include a single reflective surface 130-a positioned on an outer surface of the layer of glass 125-a. In some cases, the reflective surface 130-a may be positioned on an inner surface of the layer of glass 125-a. The reflective surface 130-a may be positioned over the light source 115 such that the first portion of light 140 emitted from the light source 115 may pass through the layer of glass 125-a, transmit through the layer of glass 125-a into the tissue of the user, and/or reflect off the reflective surface 130-a back towards the light source 115. By positioning the reflective surface 130-a (e.g., a mirror coating) on top of the light source 115, a portion of the first portion of light 145 (e.g., reflected light) may be reflected back to the light source 115. The portion of the first portion of light 145 reflected off the reflective surface 130-a may pass back through the layer of glass 125-a towards the light source 115, the flexible PCB 150, an additional reflective surface 135-a, or a combination thereof.

The additional reflective surface 135-a may be positioned on the flexible PCB 150. The additional reflective surface 135-a may be positioned adjacent to and on either side of the light source 115. In such cases, the portion of the first portion of light 145 reflected off the reflective surface 130-a may travel through the layer of glass 125-a, to the additional reflective surface 135-a, and back through the layer of glass 125-a and into the tissue and/or to the detector 120. By positioning the additional reflective surface 135-a opposite of the reflective surface 130-a, an increased amount of the portion of the first portion of light 145 may be directed towards the detectors 120, thereby increasing the signal quality and efficiency.

In some cases, the additional reflective surface 135-a may be a different material than the reflective surface 130-a. The reflective surface 130-a may be an example of a reflective material, an opaque material, a reflective coating, a diffuse white coating, or a combination thereof. The additional reflective surface 135-a may be an example of a reflective material, an opaque material, a reflective coating, a diffuse white coating, or a combination thereof.

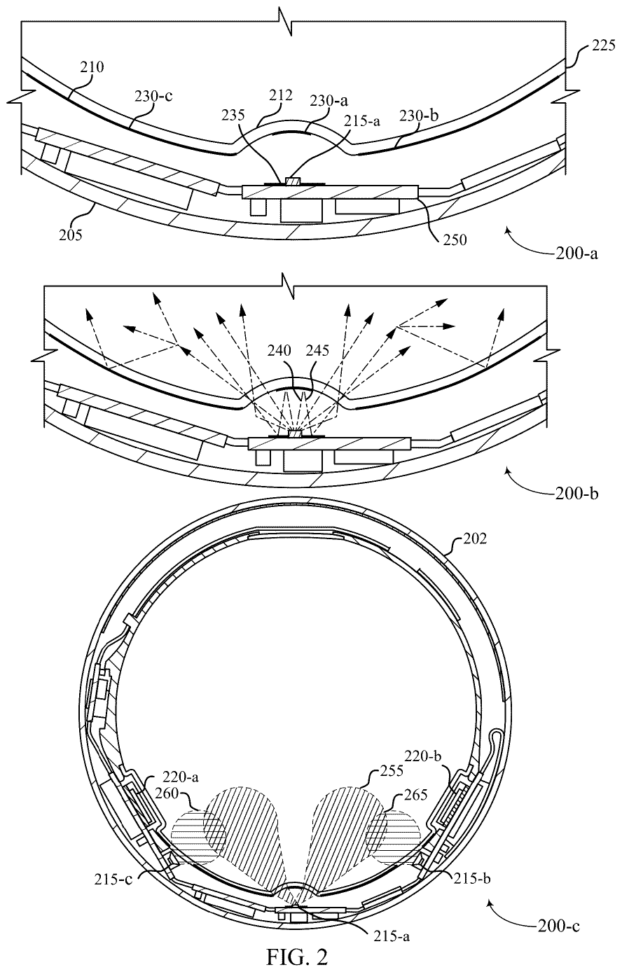

With reference to wearable device diagram 100-a, the reflective surface 130-a may be an example of a metallic or dielectric mirror coating on the layer of glass 125-a, and the additional reflective surface 135-a may be an example of a diffuse, reflective, white coating on the flexible PCB 150. The reflective surface 130-a, the additional reflective surface 135-a, or both may be used for light source 115 emission pattern modification, as described herein with reference to FIG. 2.

Wearable device diagram 100-b may include a layer of glass 125-b that includes a partially-domed portion 175. The partially-domed portion 175 may be positioned over the light source 115. By adding shapes (e.g., a partially-domed portion 175) to the layer of glass 125-b with the reflective surfaces 130-b, the light emission patterns emitted from the light source 115 may have improved efficiency as compared to wearable device diagram 100-a.

The layer of glass 125-b may include a single reflective surface 130-b positioned on the outer surface of the layer of glass 125-b. For example, reflective surface 130-b may be adhered to the top of the partially-domed portion 175 of the layer of glass 125. In some cases, the reflective surface 130-b may be positioned on the inner surface of partially-domed portion 175 of the layer of glass 125-b.

The wearable device diagram 100-b may include additional reflective surfaces 135-b positioned on either side of the light source 115 on the flexible PCB 150. In such cases, the portion of the first portion of light 145 reflected off the reflective surface 130-b may travel back through the layer of glass 125-b to the additional reflective surface 135-b and then back through the layer of glass 125-b and into the tissue and/or to the detector 120. By positioning the additional reflective surface 135-b opposite of the reflective surface 130-b, an increased amount of the portion of the first portion of light 145 may be directed towards the detectors 120. In some cases, the portion of the first portion of light 145 may be reflected more easily due to the partially-domed portion 175 including the reflective surface 130-b, thereby enabling the portion of the first portion of light 145 to be directed to the detectors 120, the tissue, or both more accurately.

The additional reflective surface 135-b may be a same material as the reflective surface 130-b. With reference to wearable device diagram 100-b, the reflective surface 130-b may be an example of a metallic or dielectric mirror coating on the layer of glass 125-b, and the additional reflective surface 135-b may be an example of a metallic or dielectric mirror coating on the flexible PCB 150. By using a combination of reflective coatings (e.g., reflective surface 130-b and additional reflective surface 135-b) as well as a molded shape of the layer of glass 125-b (e.g., including the partially-domed portion 175), a light emission pattern of the light source 115 may be modified efficiently, as described herein with reference to FIG. 2.

Wearable device diagram 100-b may include a titanium oxide material 180. The titanium oxide material 180 may be disposed within the layer of glass 125-b. In some cases, a laser may be applied to a portion of the layer of the glass 125-b such that the titanium oxide material 180 within the layer of glass 125-b may change properties In such cases, the portion of the layer of glass 125-b where the laser is applied may change from a transparent color to an opaque (e.g., dark) color due to a laser activation process. The quantity of titanium oxide material 180 disposed within the layer of glass 125-b may be mixed with the material of the layer of glass 125-b such that the light absorption in the area where the laser is not applied may not be affected (e.g., the color may remain transparent without changing to the opaque color).

The portion of the layer of the glass 125-b where the titanium oxide material 180 changes from the transparent color to the opaque color may prevent light transmission inside the layer of glass 125-b. For example, the titanium oxide material 180 activated by the laser may prevent (e.g. block) the first portion of the light 145 from entering the layer of glass 125-b where the titanium oxide material 180 has changed to an opaque color. The portion of the layer of glass 125-b where the titanium oxide material 180 changes properties may be disposed within the layer of glass 125-b that is adjacent to the partially-domed portion 175 (e.g., including the reflective surfaces 130-b). In some cases, the titanium oxide material 180 may be disposed within the layer of glass 125-b that does not include the partially-domed portion 175, the reflective surfaces 130-b, or any combination thereof.

Wearable device diagram 100-c may include the layer of glass 125-c. The layer of glass 125-c may include at least two reflective surfaces 130. A first reflective surface 130-c may be positioned on the outer surface of the layer of glass 125-c and a second reflective surface 130-d may be positioned on the inner surface of the layer of glass 125-c. The two reflective surfaces 130 may form a light guide along the layer of glass 125-c that may be used for light emission shifting. In such cases, the point of emission of the first portion of light 140 may be shifted away from the light source 115. For example, the wearable device diagram 100-c may include coatings (e.g., reflective surfaces 130) on both surfaces of the glass piece (e.g., the layer of glass 125-c) and the portion of the first portion of light 145 that is reflected off the first reflective surface 130-c, onto the second reflective surface 130-d, and through the light guide may exit the layer of glass 125-c a pre-determined distance from the light source 115.

The first reflective surface 130-c may be positioned over the light source 115 such that the first portion of light 140 emitted from the light source 115 may pass through the layer of glass 125-c, reflect off the first reflective surface 130-c towards the second reflective surface 130-d, and into the light guide and/or back towards the additional reflective surface 135-c. By positioning the reflective surface 130-c (e.g., a mirror coating) on top of the light source 115, the portion of the first portion of light 145 may be reflected into the light guide. The portion of the first portion of light 145 reflected off the reflective surface 130-c may pass back through the layer of glass 125-c towards the light source 115, the flexible PCB 150, an additional reflective surface 135-c, the second reflective surface 130-d, or a combination thereof.

The additional reflective surface 135-c may be positioned on the flexible PCB 150. The additional reflective surface 135-c may be positioned adjacent to the light source 115 and/or below the first reflective surface 130-c. In such cases, the portion of the first portion of light 145 reflected off the reflective surface 130-c may travel through the layer of glass 125-c, to the additional reflective surface 135-c, and back through the layer of glass 125-c and into the light guide. By positioning the additional reflective surface 135-c opposite of the reflective surface 130-c, an increased amount of the portion of the first portion of light 145 may be directed towards the light guide, thereby increasing an amount of light that reaches the detector 120 and thus increases the signal quality and efficiency.

The layer of glass 125-c may include total internal reflection surfaces 155, micro-optical structures 165, uneven surfaces 160, or a combination thereof. The total internal reflection surfaces 155, micro-optical structures 165, uneven surfaces 160, or a combination thereof may be used to spatially diffuse the first portion of light 140 through the layer of glass 125. In some cases, the internal reflection surfaces 155, the micro-optical structures 165, the uneven surfaces 160, or a combination thereof may alter the emission pattern of the light source 115.

Due to the presence of total internal reflection surfaces 155, micro-optical structures 165, uneven surfaces 160, or a combination thereof, the reflective surfaces 130 may be omitted from the portion of the layer of glass 125-c that includes the total internal reflection surfaces 155, micro-optical structures 165, uneven surfaces 160, or a combination thereof. In some cases, the total internal reflection surfaces 155, micro-optical structures 165, uneven surfaces 160, or a combination thereof may be used to couple light into the light guide.

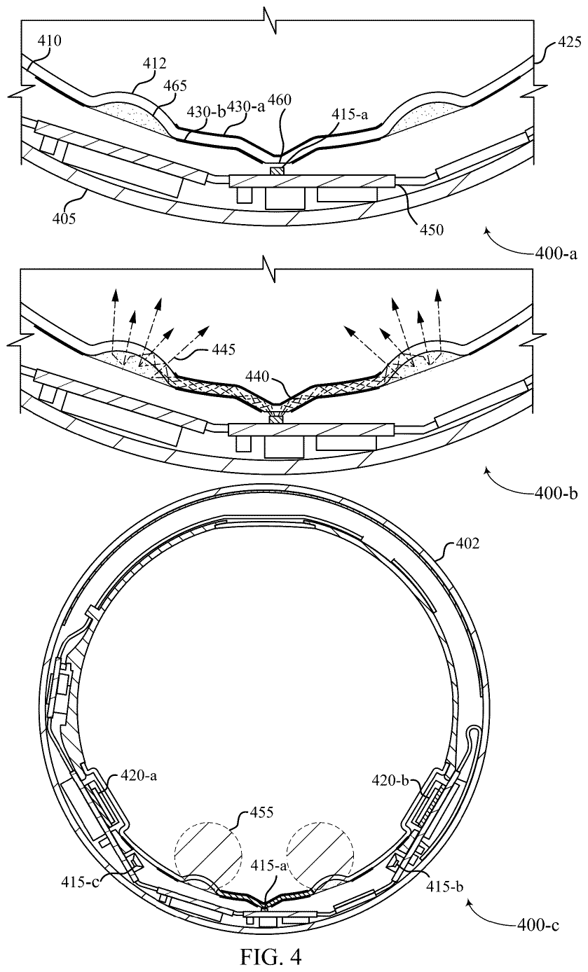

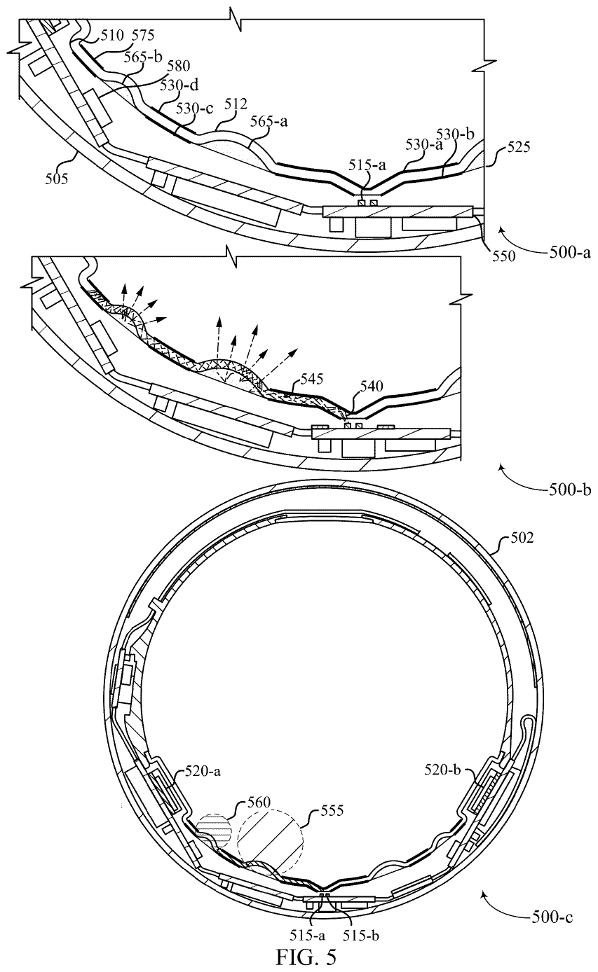

The layer of glass 125-c may include a phosphor material 170. The phosphor material 170 may be an example of a material that includes properties that changes the wavelength of the first portion of light 140 that enters the phosphor material 170 and exits the phosphor material 170. For example, the first portion of light 140 may enter the phosphor material 170 at a first wavelength and then the first portion of light 140 that exits the phosphor material 170 may include a second wavelength different than the first wavelength. The phosphor material 170 is further described herein with reference to FIGS. 4 and 5.

By measuring the signals (e.g., at detectors 120), it may be possible to use light source 115 and detector 120 pairs that have sufficient optical paths during rapid motion and reduce battery consumption. By incorporating one or more reflective surfaces 130 into the layer of glass 125, the optical efficiency of the light source 115, the detectors 120, or both may be improved. For example, the optical efficiency with respect to coupling the first portion of light 140 from the light source 115 to the detector 120 through the tissue may be increased. The portion of the first portion of light 145 may be directed through the tissue in an optical manner such that increased amounts of light may enter the detectors 120, thereby using less optical power less current, and saving battery while still maintaining good signal quality.

FIG. 2 shows an example of wearable device diagrams 200 that supports optical coatings for glass sheets in wearable devices in accordance with aspects of the present disclosure. Wearable device diagrams 200 may illustrate examples of wearable devices 202 which may be examples of wearable devices 102 with respect to FIG. 1.

With reference to wearable device diagram 200-a, the wearable device 202 may include a housing that includes an inner ring-shaped housing 210 and an outer ring-shaped housing 205. The light sources 215, detectors 220, flexible PCB 250, and other electronic circuitry may be disposed at least partially within the housing and positioned between the inner ring-shaped housing 210 and the outer ring-shaped housing 205. For example, the light sources 215 and detectors 220 may be disposed on the flexible PCB 250.

The layer of glass 225 may include a partially-domed portion 212. The layer of glass 225 may be at least partially coupled with the inner ring-shaped housing 210. For example, the layer of glass 225 may contact at least a portion of the inner ring-shaped housing 210 and extend along the inner ring-shaped housing 210 while the partially-domed portion 212 of the layer of glass 225 may be uncoupled (e.g., not in contact) with the inner ring-shaped housing 210. The partially-domed portion 212 of the layer of glass 225 may be positioned over the light source 215-a.

The layer of glass 225 may include one or more reflective surfaces 230. For example, the wearable device 202 may include at least a first reflective surface 230-a, a second reflective surface 230-b, and a third reflective surface 230-c. A first reflective surface 230-a may be adhered to the partially-domed portion 212 of the layer of glass 225. In such cases, the first reflective surface 230-a may be positioned over the light source 215-a. A second reflective surface 230-b may be adhered to layer of glass 225 adjacent to the partially-domed portion 212, and a third reflective surface 230-c may be adhered to the layer of glass 225 adjacent to the partially-domed portion on the other side of the light source 215-a. The first reflective surface 230-a adhered to the partially-domed portion 212 may be uncoupled from the inner ring-shaped housing 210 while the second reflective surface 230-b and the third reflective surface 230-c are coupled with the inner ring-shaped housing 210. In such cases, the second reflective surface 230-b and third reflective surface 230-c may be coupled between the inner ring-shaped housing 210 and the layer of glass 225 such that the second reflective surface 230-b and third reflective surface 230-c are positioned between the inner ring-shaped housing 210 and the layer of glass 225.

The reflective surfaces 230 may extend along at least a portion of the layer of glass 225 such that the one or more reflective surfaces 230 are disposed along at least a portion of an inner surface of the layer of glass 225. The inner surface of the layer of glass may be coupled with the inner ring-shaped housing 210. The reflective surface 230 may be an example of the reflective surface 130 as described with reference to FIG. 1.

With reference to wearable device diagram 200-b, the layer of glass 225 may be positioned to receive at least a first portion of light 240 emitted from the light source 215-a. For example, the layer of glass 225 may be positioned to absorb at least the first portion of light 240 emitted from the light source 215-a. In some cases, the layer of glass 225 may transmit at least the first portion of light 240 through the layer of glass 225 to be scattered and absorbed by the tissue of the user.

The reflective surfaces 230 may be positioned to reflect at least a portion of light 245. For example, the portions of the layer of glass 225 that include the reflective surfaces 230 may reflect at least the portion of the first portion of the light 245 back through the layer of glass 225 and towards the light source 215-a.

In some cases, the wearable device 202 may include one or more additional reflective surfaces 235 coupled to the flexible PCB 250. The one or more additional reflective surfaces 235 may be an example of the one or more additional reflective surfaces 135 as described with reference to FIG. 1. The portion of the first portion of the light 245 may continue to be reflected back through the layer of glass 225 such that the portion of the first portion of the light 245 may then be reflected by the one or more additional reflective surfaces 235 and through the layer of glass 225 and into the tissue of the user.

When the light source 215-a emits the first portion of light 240, some of the light (e.g., a portion of light 245) is reflected back to the light source 215-a and some of the light (e.g., the first portion of light 240) travels into the tissue through the partially-domed portion 212. The reflective surfaces 230 may also be adhered to the layer of glass 225 next to the partially-domed portion 212 so that the first portion of light 240 that propagates inside the tissue may be scattered back towards the layer of glass and the reflective surfaces 230 (e.g., the second reflective surface 230-b and the third reflective surface 230-d). In such cases, the first portion of light 240 may be recycled. The portion of the first portion of the light 245 and the first portion of light 240 may be transmitted though the layer of glass 225 on either side of the partially-domed portion 212 and may be reflected back towards the light source 215-a at the partially-domed portion 212.

With reference to wearable device diagram 200-c, the wearable device 202 may include light sources 215, which may emit light received by detector 220-a and/or detector 220-b. In this regard, the light sources 215 may support one or more optical paths through the tissue for physiological data measurements. For instance, the light source 215-a may support an optical path between the light source 215-a and the detector 220-a and another optical path between the light source 215-a and the detector 220-b. In some cases, the light source 215-a may be a red and infrared LED, which may emit light that is scattered and absorbed by the tissue of a user of the wearable device 202.

Similarly, the wearable device 202 may include light source 215-b and light source 215-c. For example, the light source 215-b may emit light. The light source 215-b and the light source 215-c may be green LEDs. The light may be scattered and absorbed by the tissue of the user, and measured via the detectors 220-a and/or 220-b. As noted previously herein, each of the light sources 215-b and 215-c may support one or more optical paths via the respective detectors 220-a and 220-b. For instance, the light source 215-b may support an optical path between the light source 215-b and the detector 220-b and another optical path between the light source 215-b and the detector 220-a. The light source 215-c may support an optical path between the light source 215-c and the detector 220-b and another optical path between the light source 215-c and the detector 220-a.

The detectors 220-a and 220-b may be configured to measure light from the respective light sources 215 which is reflected by the tissue and/or transmitted through the tissue (e.g., reflective and/or transmissive measurements). In such cases, the light may be used for physiological data measurements associated with the user. The detectors 220 may be configured to receive at least the portion of the first portion light 245 reflected from the reflective surfaces 230. In some cases, the detectors 220 may be configured to receive at least the first portion of light 240 emitted from the light sources 215.

In some examples, the inner ring-shaped housing 210 may include a dome structure over the one or more light sources 115, one or more detectors 120, or both. For example, the wearable device 202 may include dome structures over the light source 215-a. In such cases, the layer of glass 225 may include the partially-domed portion 212 over the light source 215-a. The wearable device 202 may use the light propagation from the light sources 215 to the detectors 220 through the tissue and along the one or more optical paths for physiological measurements, such as PPG and SpO2 measurements. That is, the wearable device 202 may use the light from the light source 215-a, which may include red and infrared wavelengths, to measure SpO2 and the light from the light source 215-b or light source 215-c, which may include green wavelengths, to measure PPG.

In some examples, the wearable device 202 may be subjected to a force or an acceleration, causing an air gap between the surface of the tissue and one or more sensors at the wearable device 202. The air gap between the tissue and the light sources 215 and the detectors 220 may disturb the optical paths, as light may be coupled to the tissue through two interfaces (e.g., the interface between the light sources 215 and the detectors 220 and the air and the interface between the air and the tissue). Additionally or alternatively, liquid or other contaminants may be trapped between the tissue and the light sources 215 and the detectors 220. The contaminants may dampen or absorb the optical signals. Further, the difference between refractive indexes and contaminant layer absorption spectra may determine how different signal paths/channels may be affected (e.g., causing increased variability in signal strength).

In some examples, the reflective surfaces 230 may be molded from a material (e.g., metal and the like) that is capable of reflecting light. That is, the reflective surfaces 230 may have optical properties that allow the reflective surfaces 230 to propagate a portion of light 245 from the light sources 215 to the detectors 220 with modified light emission patterns 255. The reflective surfaces 230 may be configured to alter the light pattern of the light emitted from the light sources 215. For example, the reflective surfaces 230 may be configured to manipulate a light emission direction.

With reference to wearable device diagram 200-c, the light source 215-a may include a light emission pattern 255. The light emission pattern 255 may include a beam width (e.g., a light emission size), a beam shape (e.g., a light emission shape), a beam direction (e.g., light emission direction), a beam angle (e.g., a light emission tilt angle), or a combination thereof. The light sources 215-b and 215-c may include a light emission pattern 260. The light emission pattern 260 may include a beam width (e.g., a light emission size), a beam shape (e.g., a light emission shape), a beam direction (e.g., light emission direction), a beam angle (e.g., a light emission tilt angle), or a combination thereof.

Without the reflective surfaces 230, the light emission pattern 255 may include a uniform pattern of even distribution that is direct towards the center of the tissue which may be inefficient for physiological data measurements. However, with the use of the reflective surfaces 230, the light emission pattern 255 may be directed into at least two directions (e.g., two halves) to form a heart-shaped light emission pattern. The light may be directed towards the sides of the tissue and towards the detectors 220 rather than the center of the tissue (e.g., middle of the finger). By modifying the light emission pattern 255, the signal quality may increase and the overall efficiency of the wearable device 202 may increase. The light emission pattern 255 may be directed into a plurality of directions to form a plurality of shapes. That is, the light may be emitted through the portion of the layer of glass 225 where the reflective surfaces 230 are not positioned.

By including the reflective surfaces 230 in the wearable device 102 (e.g., over the light source 215-a), the light emission pattern 255 may be modified to direct light deeper into the tissue and directed towards the detectors 220. In such cases, less stray light propagating both inside the ring structure and in the superficial layers of the skin may be emitted, and the light received by the detectors 220 may be from light coming from deeper structures within the tissue. The tilt direction of the light emission pattern 255 may be modified towards the detectors 220 in order to contribute additional light distributions to the detectors 220. By directing more light into the direction of the detectors 220 by the use of the reflective surface 230, the detectors 220 may operate at higher efficiency of the signal. In some cases, the light emission pattern 255 may be modified to form an overlapping portion 265 with the light emission pattern 260 from the light sources 215-b and 215-c.

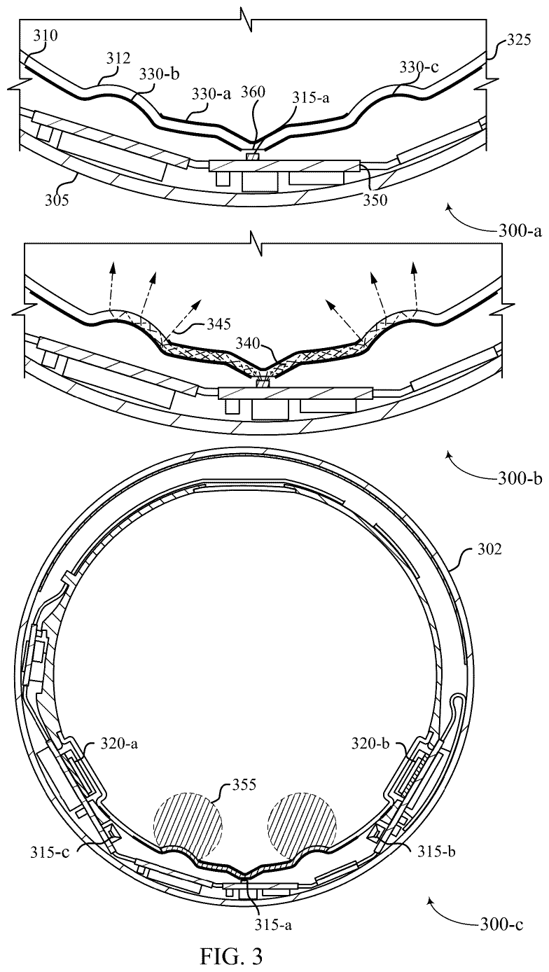

FIG. 3 shows an example of wearable device diagrams 300 with a light guide that supports optical coatings for glass sheets in wearable devices in accordance with aspects of the present disclosure. Wearable device diagrams 300 may illustrate examples of wearable devices 102 which may be examples of wearable devices 102 with respect to FIGS. 1 and 2.

With reference to wearable device diagram 300-a, the wearable device 302 may include a housing that includes an inner ring-shaped housing 310 and an outer ring-shaped housing 305. The light sources 315, flexible PCB 350, and other electronic circuitry may be disposed at least partially within the housing and positioned between the inner ring-shaped housing 310 and the outer ring-shaped housing 305.

The layer of glass 325 may include at least two partially-domed portions 312. Portions of the layer of glass 325 may be coupled with (e.g., contact) the inner ring-shaped housing 310 while the partially-domed portions 312 of the layer of glass 325 may be uncoupled (e.g., not in contact) with the inner ring-shaped housing 310. The partially-domed portions 312 may be positioned a predefined distance away from the light source 315-a to direct the first portion of light 340 emitted from the light source 315-a towards the detectors 320.

The layer of glass 325 may include one or more reflective surfaces 330. The one or more reflective surfaces 330 are adhered along an inner surface of the layer of glass 325 (e.g., adjacent to the inner ring-shaped housing) and an outer surface of the layer of glass 325 (e.g., adjacent to the tissue surface) to form a light guide that reflects the first portion of light 340 between the inner surface and the outer surface and along the layer of glass 325.

For example, the wearable device 202 may include at least three reflective surfaces 330. A first reflective surface 330-a may be disposed along the outer surface of the layer of glass 325, and a second reflective surface 330-b may be disposed along an inner surface of the layer of glass 325 opposite the outer surface. The first reflective surface 330-a may be positioned over the light source 215-a. The second reflective surface 330-b may extend along the inner surface of the layer of glass 325 such that the second reflective surface 330-b is positioned on an inner surface of the partially-domed portion 312 of the layer of glass 325. The wearable device 302 may include a third reflective surface 330-c that is the same as the second reflective surface 330-b.

In some cases, the reflective surfaces 330 on both surfaces of the layer of glass 325 may form a light guide. The light guide may extend along the layer of glass 325 in both directions from the light source 315-a and towards the partially-domed portions 312 of the layer of glass 325. The reflective surfaces 330 on both surfaces of the layer of glass 325 may enable the first portion of light 340 that enters the layer of glass 325 to bounce between the reflective surfaces 330 (e.g., the first reflective surface 330-a and the second reflective surface 330-b, for example) and along the light guide until the light reaches the partially-domed portion 312 where the portion of the first portion of light 345 may reflect off the second reflective surface 330-b and into the tissue.

In some cases, the wearable device 302 may include a microprism 360. The microprism 360 may be coupled to the layer of glass 325 and configured to couple the first portion of light 340 into the layer of glass 325. The microprism 360 may couple the portion of the first portion of light 345 into the light guide. The microprism 360 may be glued to the inner surface of the layer of glass 325. In some cases, the microprism 360 may be formed into the layer of glass 325 in a molding process. In some cases, the layer of glass 325 may be melted onto a mold that already has the microprism 360 in the structure. In some cases, the wearable device 302 may omit the additional reflective surfaces, as compared with the wearable device 202 of FIG. 2, due to the presence of the microprism, the light guide, or both.

With reference to wearable device diagram 300-b, the layer of glass 325 may be positioned to receive at least the first portion of light 340 emitted from the light source 315-a. For example, the layer of glass 325 may be positioned to absorb at least the first portion of light 340 emitted from the light source 315-a. In some cases, the layer of glass 325 may transmit at least the portion of the first portion of light 345 through the layer of glass 325 to be scattered and absorbed by the tissue of the user.

The light guide may include reflective coatings (e.g., reflective surfaces 330) on both sides of the layer of glass 325. The light may be coupled into the light guide by first having the first portion of light 340 travel through the layer of glass 325, reflect the portion of the first portion of light 345 off the first reflective surface 330-a towards the microprism 360, and then the microprism 360 reflects the portion of the first portion of light 345 back into the layer of glass 325 to be reflected back and forth off the reflective surfaces 330 along the light guide. The partially-domed portions 312 include mirrors (e.g., reflective surfaces 330) on the bottom portions but no mirror on the top part of the partially-domed portions 312. In such cases, the portion of the first portion of light 345 may escape the light guide and exit the layer of glass 325 through the top of the partially-domed portions 312.

With reference to wearable device diagram 300-c, the wearable device 302 may include light sources 315, which may emit light received by detector 320-a and/or detector 320-b. In some cases, the light source 315-a may be a red and infrared LED, which may emit light that is scattered and absorbed by the tissue of a user of the wearable device 302. Similarly, the wearable device 302 may include light source 315-b and light source 315-c. The light source 315-b and the light source 315-c may be green LEDs.

The detectors 320-a and 320-b may be configured to measure light from the respective light sources 315 which is reflected by the tissue and/or transmitted through the tissue (e.g., reflective and/or transmissive measurements). The detectors 320 may be configured to receive at least the portion of the first portion light 345 reflected from the reflective surfaces 330 within and along the light guide.

The reflective surfaces 330 may have optical properties that allow the reflective surfaces 330 to propagate the portion of the first portion of light 345 from the light source 315-a to the detectors 320 with modified light emission patterns 355. For example, the reflective surfaces 330 may be configured to alter the light pattern of the light emitted from the light sources 315-a. In such cases, the reflective surfaces 330 may be configured to manipulate a light emission direction.

With reference to wearable device diagram 300-c, the light source 315-a may include light emission patterns 355. The light emission pattern 355 may include a beam width (e.g., a light emission size), a beam shape (e.g., a light emission shape), a beam direction (e.g., light emission direction), a beam angle (e.g., a light emission tilt angle), or a combination thereof. The portion of the first portion of light 345 may exit the light guide via the partially-domed portions 312 of the layer of glass 325. In such cases, the light emission patterns 355 may be present over the partially-domed portions 312 of the layer of glass 325. That is, the light may be emitted through the portion of the layer of glass 325 where the reflective surfaces 330 are not positioned on the upper surface of the layer of glass 325.

Without the reflective surfaces 330 and/or the light guide, the light emission pattern 355 may include a single, uniform structure of even distribution that is directed towards the center of the tissue which may be inefficient for physiological data measurements. However, with the use of the reflective surfaces 330 and/or the light guide, two light emission patterns 355 may be present with each directed into at least two directions. The light may be directed towards the sides of the tissue and towards the detectors 320 rather than the center of the tissue (e.g., middle of the finger). By modifying the light emission patterns 355, the signal quality may increase and the overall efficiency of the wearable device 302 may increase.

By including the reflective surfaces 330 in the wearable device 302 on the inner surface of the partially-domed portion 312 of the layer of glass 325, the light emission patterns 355 may be modified to direct light towards the detectors 320. The tilt direction of the light emission pattern 355 may be modified towards the detectors 320 in order to contribute additional light distributions to the detectors 320. By directing more light into the direction of the detectors 320 by the use of the reflective surface 330 and/or the light guide, the detectors 320 may operate at higher efficiency of the signal.

In some cases, the light emission patterns 355 may be directed closer towards the detectors 320 to shorten the distance between the light source 315-a and the detectors 320. In such cases, a total efficiency of the light sources 315 and the detectors 320 may be increased by increasing the quantity of light that is received at the detectors 320. In such cases, space in the ring may be saved by guiding the light on top of the ring in the layer of glass 325.

FIG. 4 shows an example of wearable device diagrams 400 with phosphor material that supports optical coatings for glass sheets in wearable devices in accordance with aspects of the present disclosure. Wearable device diagrams 400 may illustrate examples of wearable devices 402 which may be examples of wearable devices 102 with respect to FIGS. 1 through 3.

The wearable device 402 may be an example of wearable device 302 with respect to FIG. 3. For example, the wearable device 402 may include an outer ring-shaped housing 405, an inner ring-shaped housing 410, light sources 415 (e.g., light source 415-a, 415-b, and 415-c), detectors 420, layer of glass 425, flexible PCB 450, reflective surfaces 430 forming a light guide, partially-domed portions 412, and microprism 460. However, rather than including a second reflective surface 430-b on the inner surface of the partially-domed portion 412 of the layer of glass 425, the wearable device 402 may include a phosphor material 465 within the partially-domed portion 412 of the layer of glass 425.

With reference to wearable device diagram 400-a, the phosphor material 465 may be disposed within the partially-domed portion 412 of the layer of glass 425. In some cases, the phosphor material 465 may be embedded within the layer of glass 425. In other examples, the phosphor material 465 may be deposited beneath the inner surface of the layer of glass 425. The phosphor material 465 may receive the portion of the first portion of light 445 reflected through the light guide and transmit the portion of the first portion of light 445 into the tissue and to the detectors 420. The phosphor material 465 may be an example of a fluorescent material, a phosphorescence material, or both.

The phosphor material 465 may include properties that allow the portion of the first portion light 445 to enter the phosphor material 465 at a first wavelength and exit the phosphor material 465 at a second wavelength different than the first wavelength. For example, the phosphor material 465 may include properties that allow the portion of the first portion of light 445 to enter the phosphor material 465 at a first wavelength corresponding to blue light and exit the phosphor material 465 at a second wavelength corresponding to yellow light, green light, or red light. In such cases, the phosphor material 465 may absorb the portion of the first portion of light 445 at one wavelength (e.g., corresponding to blue light) and emit the portion of the first portion of the light 445 at a different wavelength (e.g., corresponding to yellow light).

The phosphor material 465 may be used with a phosphor excitation light. The phosphor excitation light may be an example of a higher-energy emitted light that is emitted from the light source 415-a and absorbed by the phosphor material 465. The phosphor material 465 may then emit lower-energy light that is used for the sensor signal. In such cases, the first portion of light 440 emitted from the light source 415-a may be more energetic than the light emitted from the phosphor material 465. For example, a green light excitation from the phosphor material 465 may be used for red light emission from the light source 415-a, and red light excitation from the phosphor material 465 may be used for IR light emission from the light source 415-a. In some cases, the emitted light range from the phosphor material 465 may be extended to SWIR short-wave infrared (SWIR) wavelengths where there may not be light sources 415 components readily available to emit light of SWIR wavelengths.

With reference to wearable device diagram 400-b, the light guide may include reflective coatings (e.g., reflective surfaces 430) on both surfaces of the layer of glass 425. The portion of the first portion of light 445 may be coupled into the light guide by first having the first portion of light 440 travel through the layer of glass 425, reflect off the top reflective surface 430-a towards the microprism 460, and then the microprism 460 reflects the portion of the first portion of light 445 back into the layer of glass 425 to be reflected back and forth off the reflective surfaces 430 along the light guide.

The partially-domed portions 412 include the phosphor material 465 that allows the portion of the first portion of light 445 to be absorbed into the phosphor material 465 at a wavelength corresponding to blue light and then emit the portion of the first portion of light 445 into the tissue at a wavelength corresponding to yellow light. In such cases, the portion of the first portion of light 445 may escape the light guide and exit the layer of glass 425 through the top portion of the partially-domed portions 412 after the phosphor material 465 converts the portion of the first portion of light 445 from the first wavelength to the second wavelength.

The phosphor material 465 may utilize fluorescence for emission wavelength shifts. The use of phosphor material 465 within the layer of glass 425 may enable additional light spectral tuning. That is, by integrating the phosphor material 465 into the layer of glass 425, the emission wavelength of the light source 415-a may be modified without having to change each component (e.g., light source) that is used for illuminating.

With reference to wearable device diagram 400-c, the wearable device 402 may include light sources 415-a, which may emit light received by detector 420-a and/or detector 420-b. In some cases, the light source 415-a may be a blue VCSEL, which may emit light that is scattered and absorbed by the tissue of a user of the wearable device 402.

The reflective surfaces 430 in combination with the phosphor material 465 may have properties that allow the reflective surfaces 430 to propagate the portion of the first portion of light 445 from the light source 415-a to the phosphor material 465 and to the detectors 420 with modified light emission patterns 455. For example, the reflective surfaces 430 may be configured to alter the light pattern of the light emitted from the light sources 415-a, and the phosphor material 465 may be configured to alter the wavelength of the light emitted from the light source 415-a. In such cases, the reflective surfaces 430 may be configured to manipulate a light emission direction, and the phosphor material 465 may be configured to manipulate a color of the light emission pattern 455.

With reference to wearable device diagram 400-c, the portion of the first portion of light 445 may exit the light guide via the partially-domed portions 412 of the layer of glass 425. In such cases, the light emission patterns 455 may be present over the partially-domed portions 412 of the layer of glass 425. That is, the portion of the first portion of light 445 may be emitted through the portion of the layer of glass 425 where the reflective surfaces 430 are not positioned on the upper surface of the layer of glass 425. The light emission pattern 455 may include a yellow light emission pattern as opposed to the blue light emission pattern expected from the light source 415-a. In such cases, the phosphor material 465 may alter the light emission pattern 455 from the blue light emission pattern to the yellow light emission pattern.

Without the reflective surfaces 430, the light guide, and/or the phosphor material 465, the light emission pattern 455 may include a single, uniform structure of even distribution corresponding to the color of the wavelength of light emitted from the light source 415-a that is directed towards the center of the tissue which may be inefficient for physiological data measurements. However, with the use of the reflective surfaces 430, the light guide, and/or the phosphor material 465, two light emission patterns 455 of colors different than the light source 15-a may be present with each directed into at least two directions. By modifying the light emission patterns 455, the signal quality may increase and the overall efficiency of the wearable device 402 may increase.

FIG. 5 shows an example of wearable device diagrams 500 with phosphor materials that supports optical coatings for glass sheets in wearable devices in accordance with aspects of the present disclosure. Wearable device diagrams 300 may illustrate examples of wearable devices 102 which may be examples of wearable devices 102 with respect to FIGS. 1 and 2.

The wearable device 502 may be an example of wearable device 402 with respect to FIG. 4. For example, the wearable device 502 may include an outer ring-shaped housing 505, an inner ring-shaped housing 510, light sources 515 (e.g., light source 515-a and 515-b), detectors 520, layer of glass 525, flexible PCB 550, reflective surfaces 530 forming a light guide, partially-domed portions 512, microprism 360, and phosphor material 565. In addition to the features described with reference to FIG. 4, the wearable device 502 may also include a second phosphor material 565-b, reflective surfaces 530-c and 530-d that extend between the first phosphor material 565-a and second phosphor material 565-b, and black coatings 575.

The black coatings 575 may be disposed along the outer surface of the layer of glass 525 and along the inner surface of the layer of glass 525 opposite the outer surface. The black coatings 575 may form a light guide that enables the portion of the first light 540 that enters the light guide to bounce between the black coatings and along the light guide until the portion of the first portion of light 545 travels back to partially-domed portion 512 where the portion of the first portion of light 545 may reflect off the second phosphor material 565-b and into the tissue. In such cases, the black coatings 575 may be used to capture the portion of the first portion of light 545 that escapes the second phosphor material 565-b portion of the first portion of light 545. For example, the black coatings 575 may absorb extra light that misses (e.g., passes) the phosphor material 565, thereby reducing an amount of stray light that reaches the detectors 520. The black coatings 575 may be used to manipulate where the portion of the first portion of light 545 goes inside the light guide and where the portion of the first portion of light 545 exits the light guide.

With reference to wearable device diagram 500-a, the reflective surfaces 530-c and 530-d that extend between the first phosphor material 565-a and second phosphor material 565-b may form a second light guide between the partially-domed portions 512 of the layer of glass 525. The reflective surfaces 530-c and 530-d that extend between the first phosphor material 565-a and second phosphor material 565-b may be disposed along the inner surface of the layer of glass 525 and the outer surface of the layer of glass 525.

The second phosphor material 565-b may be an example of the phosphor material 465 as described with reference to FIG. 4. The second phosphor material 565-b may include properties that allow the portion of the first portion of light 545 to enter the second phosphor material 565-b at a first wavelength corresponding to yellow light (e.g., from the first phosphor material 565-a) and exit the second phosphor material 565-b at a second wavelength corresponding to green light. The second phosphor material 565-b may be positioned over additional circuitry 580.

As previously described herein, the partially-domed portions 512 include the first phosphor material 565-a that allows the portion of the first portion of light 545 to be absorbed into the first phosphor material 565-a at a wavelength corresponding to blue light and then emit the portion of the first portion of light 545 into the tissue at a wavelength corresponding to yellow light. In some cases, the portion of the first portion of light 545 may be reflected into the light guide that extends between the first phosphor material 565-a and the second phosphor material 465-b. The portion of the first portion of light 545 reflected into the light guide that extends between the first phosphor material 565-a and the second phosphor material 565-b may include a wavelength corresponding to blue light. For example, blue light may be emitted from the light source 515-a, and the blue light may be converted to yellow light via the first phosphor material 565-a and/or the blue light may be converted to green light via the second phosphor material 565-b.

In some cases, the blue light in the lightguide may bypass the first phosphor material 565-a (e.g., from the sides). In such cases, the blue light may travel down the light guide, reflecting off the reflective surfaces 530, and enter the second phosphor material 565-b. The second phosphor material 565-b may absorb the light wavelength corresponding to blue light and then emit the portion of the first portion of light 545 into the tissue at a wavelength corresponding to green light. In such cases, the portion of the first portion of light 545 may escape the second phosphor material 565-b and exit the layer of glass 525 through the top of the partially-domed portions 512 after the second phosphor material 565-b converts the portion of the first portion of light 545 from a first wavelength to a second wavelength. The yellow light emitted from the first phosphor material 465-a that travels to the second phosphor material 465-b may be unable to excite the second phosphor material 465-b as wavelengths corresponding to yellow light are less energetic than wavelengths corresponding to green light.

With reference to wearable device diagram 500-c, the reflective surfaces 530 may be configured to alter the light pattern of the light emitted from the light sources 515-a, and the phosphor materials 565 may be configured to alter the wavelengths of the light emitted from the light source 515-a. In such cases, the reflective surfaces 530 may be configured to manipulate a light emission direction, and the phosphor materials 565 may be configured to manipulate a color of the light emission pattern 555 and 560.

The light emission pattern 555 may be present over the partially-domed portions 512 of the layer of glass 525 that includes the first phosphor material 565-a, and the light emission pattern 560 may be present over the partially-domed portion 512 of the layer of glass that includes the second phosphor material 565-b. The light emission pattern 555 may correspond to yellow light emitted from the first phosphor material 565-a, and the light emission pattern 560 may correspond to the green light emitted from the second phosphor material 565-b. In such cases, the light emission pattern 555 may include a yellow light emission pattern as opposed to the blue light emission pattern expected from the light source 515-a. The light emission pattern 560 may include a green light emission pattern as opposed to the blue light emission pattern expected from the light source 515-a. The first phosphor material 565-a may alter the light emission pattern 555 from the blue light emission pattern to the yellow light emission pattern, and the second phosphor material 565-b may alter the light emission pattern 560 from the yellow light emission pattern to the green light emission pattern.

By using a second phosphor material 565-b (e.g., including properties that emits light at a different wavelength than the first phosphor material 565-a), a reduced quantity of light sources 515 may be implemented within the wearable device 502. In such cases, a single light source (e.g., light source 515-a emitting blue light) may be used to generate different color light emission patterns (e.g. light emission pattern 555 and light emission pattern 560).

In some cases, the light emission pattern 560 corresponding to the green light may be positioned closer to the detectors 520 as opposed to the light emission pattern 555 corresponding to the yellow light. The green light may penetrate the tissue to a shallower depth as compared to the yellow light. In such cases, it may be beneficial to position the second phosphor material 565-a closer to the detectors 520 and position the first phosphor material 565-a closer to the light source 515-a.

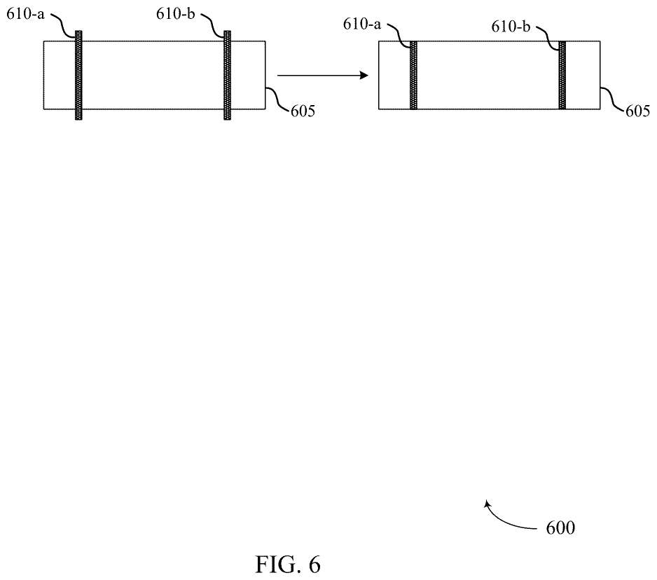

FIG. 6 shows an example of a wearable device diagram 600 with one or more metal wires that supports optical coatings for glass sheets in wearable devices in accordance with aspects of the present disclosure. Wearable device diagram 600 may illustrate examples of wearable devices 102 which may be examples of wearable devices 102 with respect to FIGS. 1 and 2.

Wearable device diagram 600 may include a layer of glass 605. The layer of glass 605 may be an example of the layer of glass as described with respect to FIGS. 1 through 5. The layer of glass 605 may include one or more metallic wires 610. For example, the layer of glass 605 may include a first metallic wire 610-a and a second metallic wire 610-b opposite of the first metallic wire 610-a. The one or more metallic wires 610 may be placed within the layer of glass 605 when the layer of glass 605 is heated to a temperature such that the one or more metallic wires 610 may be disposed within the layer of glass 605. For example, the ends of the one or more metallic wires 610 may extend through and protrude out of the layer of glass 605. The one or more metallic wires 610 may undergo a grinding procedure such that the ends that protrude out of the layer of glass 605 may be even (e.g., flush) with the surface of the layer of glass 605.

The one or more metallic wires 610 may be configured to prevent the first portion of light from entering the portion of the layer of glass 605 where the one or more metallic wires 610 are disposed. For example, the one or more metallic wires 610 may be positioned such that the first portion of light emitted from the light source may pass through the layer of glass 605 and/or reflect off the one or more metallic wires 610 and back towards the light source, the detectors, or through the layer of glass 605 and into the tissue of the user. By positioning the one or more metallic wires 610 though the layer of glass 605, an increased amount of the portion of the first portion of light may be directed towards the detectors, thereby increasing the signal quality and efficiency. In some cases, the one or more metallic wires 610 may provide through-glass vias for electrical sensor pads, block glass-internal stray light from reaching the detectors, re-direct light emitted from the light sources to enhance output, or any combination thereof.

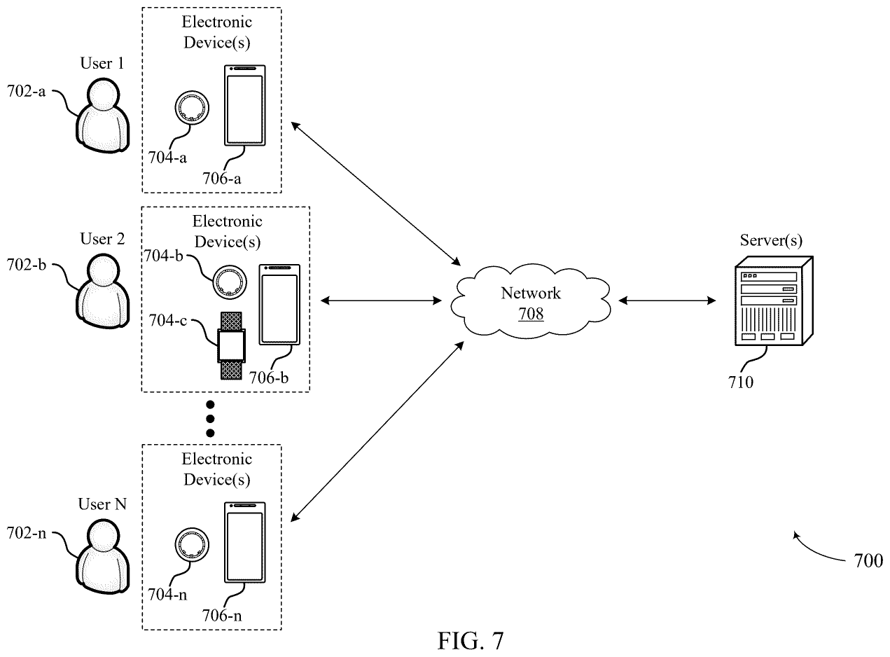

FIG. 7 illustrates an example of a system 700 that supports optical coatings for glass sheets in wearable devices in accordance with aspects of the present disclosure. The system 700 includes a plurality of electronic devices (e.g., wearable devices 704, user devices 706) that may be worn and/or operated by one or more users 702. The system 700 further includes a network 708 and one or more servers 710.

The electronic devices may include any electronic devices known in the art, including wearable devices 704 (e.g., ring wearable devices, watch wearable devices, etc.), user devices 706 (e.g., smartphones, laptops, tablets). The electronic devices associated with the respective users 702 may include one or more of the following functionalities: 1) measuring physiological data, 2) storing the measured data, 3) processing the data, 4) providing outputs (e.g., via GUIs) to a user 702 based on the processed data, and 5) communicating data with one another and/or other computing devices. Different electronic devices may perform one or more of the functionalities.

Example wearable devices 704 may include wearable computing devices, such as a ring computing device (hereinafter “ring”) configured to be worn on a user's 702 finger, a wrist computing device (e.g., a smart watch, fitness band, or bracelet) configured to be worn on a user's 702 wrist, and/or a head mounted computing device (e.g., glasses/goggles). Wearable devices 704 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 704 may also be attached to, or included in, articles of clothing. For example, wearable devices 704 may be included in pockets and/or pouches on clothing. As another example, wearable device 704 may be clipped and/or pinned to clothing, or may otherwise be maintained within the vicinity of the user 702. Example articles of clothing may include, but are not limited to, hats, shirts, gloves, pants, socks, outerwear (e.g., jackets), and undergarments. In some implementations, wearable devices 704 may be included with other types of devices such as training/sporting devices that are used during physical activity. For example, wearable devices 704 may be attached to, or included in, a bicycle, skis, a tennis racket, a golf club, and/or training weights.

Much of the present disclosure may be described in the context of a wearable device 704, which may include finger-worn wearable devices, wrist-worn wearable devices, and the like. Accordingly, the terms “wearable device 704,” “wearable ring device,” “ring,” and like terms, may be used interchangeably, unless noted otherwise herein. However, the use of the terms “wearable ring device” and/or “ring” are not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 706 may include handheld mobile computing devices, such as smartphones and tablet computing devices. User devices 706 may also include personal computers, such as laptop and desktop computing devices. Other example user devices 706 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 706 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 704, user devices 706) may measure physiological parameters of respective users 702, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 704), mobile device application, or a server computing device may process received physiological data that was measured by other devices.

In some implementations, a user 702 may operate, or may be associated with, multiple electronic devices, some of which may measure physiological parameters and some of which may process the measured physiological parameters. In some implementations, a user 702 may have a ring (e.g., wearable device 704) that measures physiological parameters. The user 702 may also have, or be associated with, a user device 706 (e.g., mobile device, smartphone), where the wearable device 704 and the user device 706 are communicatively coupled to one another. In some cases, the user device 706 may receive data from the wearable device 704 and perform some/all of the calculations described herein. In some implementations, the user device 706 may also measure physiological parameters described herein, such as motion/activity parameters.

For example, as illustrated in FIG. 7, a first user 702-a (User 1) may operate, or may be associated with, a wearable device 704-a (e.g., wearable ring device) and a user device 706-a that may operate as described herein. In this example, the user device 706-a associated with user 702-a may process/store physiological parameters measured by the wearable device 704-a. Comparatively, a second user 702-b (User 2) may be associated with wearable devices 704-b and 704-c (e.g., wearable ring device and a wrist-worn wearable device, such as a watch) and a user device 706-b, where the user device 706-b associated with user 702-b may process/store physiological parameters measured by the wearable devices 704-b and 704-c. Moreover, an nth user 702-n (User N) may be associated with an arrangement of electronic devices described herein (e.g., wearable device 704-n, user device 706-n). In some aspects, wearable devices 704 (e.g., wearable ring devices, wrist-worn wearable devices) and other electronic devices may be communicatively coupled to the user devices 706 of the respective users 702 via Bluetooth, Wi-Fi, and other wireless protocols. Moreover, in some cases, the wearable device 704 and the user device 706 may be included within (or make up) the same device. For example, in some cases, the wearable device 704 may be configured to execute an application associated with the wearable device 704, and may be configured to display data via a GUI.

In some implementations, the wearable devices 704 (e.g., wearable ring devices) of the system 700 may be configured to collect physiological data from the respective users 702 based on arterial blood flow within the user's finger. In particular, a wearable ring device may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 700 may be configured to collect physiological data from the respective users 702 based on blood flow diffused into a microvascular bed of skin with capillaries and arterioles. For example, the system 700 may collect PPG data based on a measured amount of blood diffused into the microvascular system of capillaries and arterioles. In some implementations, the wearable device 704 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the wearable device 704 (e.g., around an inner surface of the wearable ring device) has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a wearable ring device has been found to exhibit superior performance as compared to wearable devices worn on the wrist, as the wearable ring device may have greater access to arteries (as compared to capillaries), thereby resulting in stronger signals and more valuable physiological data.

The electronic devices of the system 700 (e.g., user devices 706, wearable devices 704) may be communicatively coupled to one or more servers 710 via wired or wireless communication protocols. For example, as shown in FIG. 7, the electronic devices (e.g., user devices 706) may be communicatively coupled to one or more servers 710 via a network 708. The network 708 may implement transfer control protocol and internet protocol (TCP/IP), such as the Internet, or may implement other network 708 protocols. Network connections between the network 708 and the respective electronic devices may facilitate transport of data via email, web, text messages, mail, or any other appropriate form of interaction within a computer network 708. For example, in some implementations, the wearable device 704-a associated with the first user 702-a may be communicatively coupled to the user device 706-a, where the user device 706-a is communicatively coupled to the servers 710 via the network 708. In additional or alternative cases, wearable devices 704 (e.g., wearable ring devices, wrist-worn wearable devices such as watches) may be directly communicatively coupled to the network 708.

The system 700 may offer an on-demand database service between the user devices 706 and the one or more servers 710. In some cases, the servers 710 may receive data from the user devices 706 via the network 708, and may store and analyze the data. Similarly, the servers 710 may provide data to the user devices 706 via the network 708. In some cases, the servers 710 may be located at one or more data centers. The servers 710 may be used for data storage, management, and processing. In some implementations, the servers 710 may provide a web-based interface to the user device 106 via web browsers.

In some aspects, the system 700 may detect periods of time that a user 702 is asleep, and classify periods of time that the user 702 is asleep into one or more sleep stages (e.g., sleep stage classification). For example, as shown in FIG. 7, User 702-a may be associated with a wearable device 704-a (e.g., wearable ring device) and a user device 706-a. In this example, the wearable device 704-a may collect physiological data associated with the user 702-a, including temperature, heart rate, HRV, respiratory rate, and the like. In some aspects, data collected by the wearable device 704-a may be input to a machine learning classifier, where the machine learning classifier is configured to determine periods of time that the user 702-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 702-a via a GUI of the user device 706-a. Sleep stage classification may be used to provide feedback to a user 702-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 700 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 702-a via the wearable device 704-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 702 to generate tailored, individualized circadian rhythm adjustment models that are specific to each respective user 702.

In some aspects, the system 700 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

It should be appreciated by a person skilled in the art that one or more aspects of the disclosure may be implemented in a system 700 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

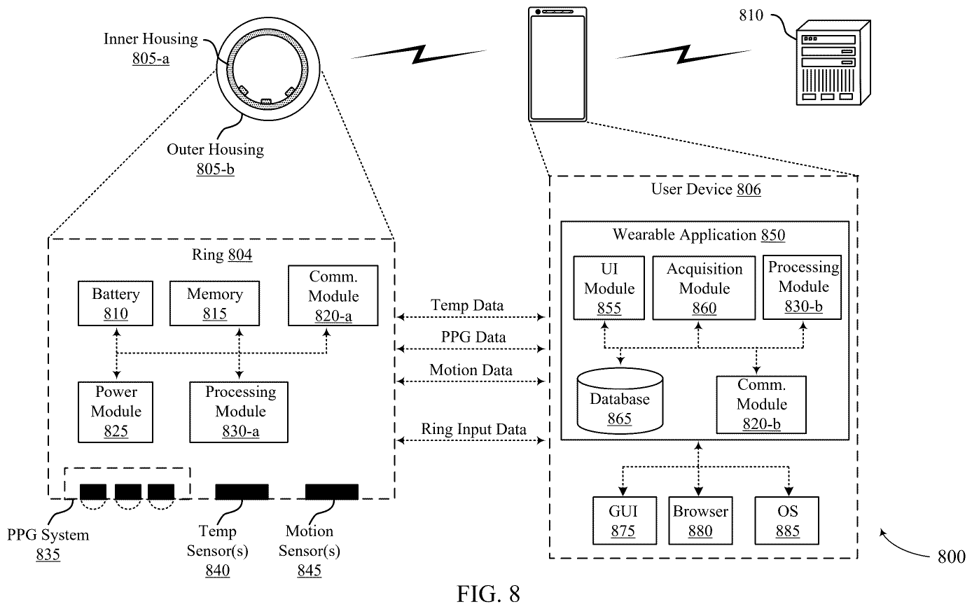

FIG. 8 illustrates an example of a system 800 that supports optical coatings for glass sheets in wearable devices in accordance with aspects of the present disclosure. The system 800 may implement, or be implemented by, system 700. In particular, system 800 illustrates a wearable device 804 (e.g., wearable ring device), a user device 806, and a server 810, as described with reference to FIG. 7.

In some aspects, the wearable device 804 (e.g., wearable ring device) may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 800 further includes a user device 806 (e.g., a smartphone) in communication with the wearable device 804. For example, the wearable device 804 may be in wireless and/or wired communication with the user device 806. In some implementations, the wearable device 804 may send measured and processed data (e.g., temperature data, photoplethysmogram (PPG) data, motion/accelerometer data, ring input data, and the like) to the user device 806. The user device 806 may also send data to the wearable device 804, such as firmware/configuration updates. The user device 806 may process data. In some implementations, the user device 806 may transmit data to the server 810 for processing and/or storage.

The wearable device 804 may include a housing 805 that may include an inner housing 805-a and an outer housing 805-b. In some aspects, the inner housing 805-a, the outer housing 805-b, or both, may include a curved profile/surface. In particular, the housing 805 may exhibit any curved or “circumferential” profile, including a circular profile, an elliptical profile, and the like. Moreover, in some cases, the inner housing 805-a, the outer housing 805-b, or both, may include both curved (e.g., “circumferential”) and flat/planar portions. For the purposes of the present disclosure, the term “circumferential” may be used interchangeably with the term “curved” to refer to circular-shaped, elliptical-shaped, or other curved-shaped profile.

In some aspects, the housing 805 of the wearable device 804 may store or otherwise include various components of the ring including, but not limited to, device electronics, a power source (e.g., battery 811, and/or capacitor), one or more substrates (e.g., printable circuit boards) that interconnect the device electronics and/or power source, and the like. The device electronics may include device modules (e.g., hardware/software), such as: a processing module 830-a, a memory 815, a communication module 820-a, a power module 825, and the like. The device electronics may also include one or more sensors. Example sensors may include one or more temperature sensors 840, a PPG sensor assembly (e.g., PPG system 835), and one or more motion sensors 845.

The sensors may include associated modules (not illustrated) configured to communicate with the respective components/modules of the wearable device 804, and generate signals associated with the respective sensors. In some aspects, each of the components/modules of the wearable device 804 may be communicatively coupled to one another via wired or wireless connections. Moreover, the wearable device 804 may include additional and/or alternative sensors or other components that are configured to collect physiological data from the user, including light sensors (e.g., LEDs), oximeters, and the like.

The wearable device 804 shown and described with reference to FIG. 8 is provided solely for illustrative purposes. As such, the wearable device 804 may include additional or alternative components as those illustrated in FIG. 8. Additional or alternative wearable devices 804 that provide functionality described herein may be fabricated. For example, wearable devices 804 with fewer components (e.g., sensors) may be fabricated. In a specific example, a wearable device 804 with a single temperature sensor 840 (or other sensor), a power source, and device electronics configured to read the single temperature sensor 840 (or other sensor) may be fabricated. In another specific example, a temperature sensor 840 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 840 (or other sensor). In other examples, a wearable device 804 that includes additional sensors and processing functionality may be fabricated.

The housing 805 may include one or more housing components. The housing 805 may include an outer housing 805-b component (e.g., a shell) and an inner housing 805-a component (e.g., a molding). The housing 805 may include additional components (e.g., additional layers) not explicitly illustrated in FIG. 8. For example, in some implementations, the wearable device 804 may include one or more insulating layers that electrically insulate the device electronics and other conductive materials (e.g., electrical traces) from the outer housing 805-b. The housing 805 may provide structural support for the device electronics, battery 811, substrate(s), and other components. For example, the housing 805 may protect the device electronics, battery 811, and substrate(s) from mechanical forces, such as pressure and impacts. The housing 805 may also protect the device electronics, battery 811, and substrate(s) from water and/or other chemicals.

The inner housing 805-a may be configured to interface with the user's finger. The inner housing 805-a may be formed from a polymer (e.g., a medical grade polymer) or other material. In some implementations, the inner housing 805-a may be transparent. For example, the inner housing 805-a may be transparent to light emitted by the PPG LEDs. In some implementations, the inner housing 805-a component may be molded onto the outer housing 805-b. For example, the inner housing 805-a may include a polymer that is molded (e.g., injection molded) to fit into an outer housing 805-b metallic shell.

The inner housing 805-a and the outer housing 805-b may be fabricated from one or more materials. In some implementations, the inner housing 805-a, the outer housing 805-b, or both, may include a metal, such as titanium, that may provide strength and abrasion resistance at a relatively light weight. Additionally, or alternatively, the inner housing 805-a, and/or the outer housing 805-b may also be fabricated from other materials, such polymers, plastic materials, epoxy materials, ceramic materials, and the like. In some implementations, the outer housing 805-b may be protective as well as decorative.

The wearable device 804 may include one or more substrates (not illustrated). The device electronics and battery 811 may be included on the one or more substrates. For example, the device electronics and battery 811 may be mounted on one or more substrates. Example substrates may include one or more printed circuit boards (PCBs), such as flexible PCB (e.g., polyimide). In some implementations, the electronics/battery 811 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 811 to the device electronics.

The device electronics, battery 811, and substrates may be arranged in the wearable device 804 in a variety of ways. In some implementations, one substrate that includes device electronics may be mounted along the bottom of the wearable device 804 (e.g., the bottom half), such that the sensors (e.g., PPG system 835, temperature sensors 840, motion sensors 845, and other sensors) interface with the underside of the user's finger. In these implementations, the battery 811 may be included along the top portion of the wearable device 804 (e.g., on another substrate).

The various components/modules of the wearable device 804 represent functionality (e.g., circuits and other components) that may be included in the wearable device 804. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 815 (memory module) of the wearable device 804 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 815 may store any of the data described herein. For example, the memory 815 may be configured to store data (e.g., motion data, temperature data, PPG data) collected by the respective sensors and PPG system 835. Furthermore, memory 815 may include instructions that, when executed by one or more processing circuits, cause the modules to perform various functions attributed to the modules herein. The device electronics of the wearable device 804 described herein are only example device electronics. As such, the types of electronic components used to implement the device electronics may vary based on design considerations.

The functions attributed to the modules of the wearable device 804 (e.g., wearable ring device) described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 830-a of the wearable device 804 may include one or more processors (e.g., processing units), microcontrollers, digital signal processors, systems on a chip (SOCs), and/or other processing devices. The processing module 830-a communicates with the modules included in the wearable device 804. For example, the processing module 830-a may transmit/receive data to/from the modules and other components of the wearable device 804, such as the sensors. As described herein, the modules may be implemented by various circuit components. Accordingly, the modules may also be referred to as circuits (e.g., a communication circuit and power circuit).

The processing module 830-a may communicate with the memory 815. The memory 815 may include computer-readable instructions that, when executed by the processing module 830-a, cause the processing module 830-a to perform the various functions attributed to the processing module 830-a herein. In some implementations, the processing module 830-a (e.g., a microcontroller) may include additional features associated with other modules, such as communication functionality provided by the communication module 820-a (e.g., an integrated Bluetooth Low Energy transceiver) and/or additional onboard memory 815.

The communication module 820-a may include circuits that provide wireless and/or wired communication with the user device 806 (e.g., communication module 820-b of the user device 806). In some implementations, the communication modules 820-a, 820-b may include wireless communication circuits, such as Bluetooth circuits and/or Wi-Fi circuits. In some implementations, the communication modules 820-a, 820-b can include wired communication circuits, such as Universal Serial Bus (USB) communication circuits. Using the communication module 820-a, the wearable device 804 and the user device 806 may be configured to communicate with each other. The processing module 830-a of the ring may be configured to transmit/receive data to/from the user device 806 via the communication module 820-a. Example data may include, but is not limited to, motion data, temperature data, pulse waveforms, heart rate data, HRV data, PPG data, and status updates (e.g., charging status, battery charge level, and/or wearable device 804 configuration settings). The processing module 830-a of the ring may also be configured to receive updates (e.g., software/firmware updates) and data from the user device 806.

The wearable device 804 may include a battery 811 (e.g., a rechargeable battery 811). An example battery 811 may include a Lithium-Ion or Lithium-Polymer type battery 811, although a variety of battery 811 options are possible. The battery 811 may be wirelessly charged. In some implementations, the wearable device 804 may include a power source other than the battery 811, such as a capacitor. The power source (e.g., battery 811 or capacitor) may have a curved geometry that matches the curve of the wearable device 804. In some aspects, a charger or other power source may include additional sensors that may be used to collect data in addition to, or that supplements, data collected by the wearable device 804 itself. Moreover, a charger or other power source for the wearable device 804 may function as a user device 806, in which case the charger or other power source for the wearable device 804 may be configured to receive data from the wearable device 804, store and/or process data received from the wearable device 804, and communicate data between the wearable device 804 and the servers 810.

In some aspects, the wearable device 804 includes a power module 825 that may control charging of the battery 811. For example, the power module 825 may interface with an external wireless charger that charges the battery 811 when interfaced with the wearable device 804. The charger may include a datum structure that mates with a wearable device 804 datum structure to create a specified orientation with the wearable device 804 during charging. The power module 825 may also regulate voltage(s) of the device electronics, regulate power output to the device electronics, and monitor the state of charge of the battery 811. In some implementations, the battery 811 may include a protection circuit module (PCM) that protects the battery 811 from high current discharge, over voltage during charging, and under voltage during discharge. The power module 825 may also include electro-static discharge (ESD) protection.

The one or more temperature sensors 840 may be electrically coupled to the processing module 830-a. The temperature sensor 840 may be configured to generate a temperature signal (e.g., temperature data) that indicates a temperature read or sensed by the temperature sensor 840. The processing module 830-a may determine a temperature of the user in the location of the temperature sensor 840. For example, in the wearable device 804, temperature data generated by the temperature sensor 840 may indicate a temperature of a user at the user's finger (e.g., skin temperature). In some implementations, the temperature sensor 840 may contact the user's skin. In other implementations, a portion of the housing 805 (e.g., the inner housing 805-a) may form a barrier (e.g., a thin, thermally conductive barrier) between the temperature sensor 840 and the user's skin. In some implementations, portions of the wearable device 804 configured to contact the user's finger may have thermally conductive portions and thermally insulative portions. The thermally conductive portions may conduct heat from the user's finger to the temperature sensors 840. The thermally insulative portions may insulate portions of the wearable device 804 (e.g., the temperature sensor 840) from ambient temperature.

In some implementations, the temperature sensor 840 may generate a digital signal (e.g., temperature data) that the processing module 830-a may use to determine the temperature. As another example, in cases where the temperature sensor 840 includes a passive sensor, the processing module 830-a (or a temperature sensor 840 module) may measure a current/voltage generated by the temperature sensor 840 and determine the temperature based on the measured current/voltage. Example temperature sensors 840 may include a thermistor, such as a negative temperature coefficient (NTC) thermistor, or other types of sensors including resistors, transistors, diodes, and/or other electrical/electronic components.

The processing module 830-a may sample the user's temperature over time. For example, the processing module 830-a may sample the user's temperature according to a sampling rate. An example sampling rate may include one sample per second, although the processing module 830-a may be configured to sample the temperature signal at other sampling rates that are higher or lower than one sample per second. In some implementations, the processing module 830-a may sample the user's temperature continuously throughout the day and night. Sampling at a sufficient rate (e.g., one sample per second) throughout the day may provide sufficient temperature data for analysis described herein.

The processing module 830-a may store the sampled temperature data in memory 815. In some implementations, the processing module 830-a may process the sampled temperature data. For example, the processing module 830-a may determine average temperature values over a period of time. In one example, the processing module 830-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 815 may store the average temperature values over time. In some implementations, the memory 815 may store average temperatures (e.g., one per minute) instead of sampled temperatures in order to conserve memory 815.

The sampling rate, which may be stored in memory 815, may be configurable. In some implementations, the sampling rate may be the same throughout the day and night. In other implementations, the sampling rate may be changed throughout the day/night. In some implementations, the wearable device 804 may filter/reject temperature readings, such as large spikes in temperature that are not indicative of physiological changes (e.g., a temperature spike from a hot shower). In some implementations, the wearable device 804 may filter/reject temperature readings that may not be reliable due to other factors, such as excessive motion during exercise (e.g., as indicated by a motion sensor 845).

The wearable device 804 (e.g., communication module) may transmit the sampled and/or average temperature data to the user device 806 for storage and/or further processing. The user device 806 may transfer the sampled and/or average temperature data to the server 810 for storage and/or further processing.

Although the wearable device 804 is illustrated as including a single temperature sensor 840, the wearable device 804 may include multiple temperature sensors 840 in one or more locations, such as arranged along the inner housing 805-a near the user's finger. In some implementations, the temperature sensors 840 may be stand-alone temperature sensors 840. Additionally, or alternatively, one or more temperature sensors 840 may be included with other components (e.g., packaged with other components), such as with the accelerometer and/or processor.

The processing module 830-a may acquire and process data from multiple temperature sensors 840 in a similar manner described with respect to a single temperature sensor 840. For example, the processing module 830 may individually sample, average, and store temperature data from each of the multiple temperature sensors 840. In other examples, the processing module 830-a may sample the sensors at different rates and average/store different values for the different sensors. In some implementations, the processing module 830-a may be configured to determine a single temperature based on the average of two or more temperatures determined by two or more temperature sensors 840 in different locations on the finger.

The temperature sensors 840 on the wearable device 804 (e.g., wearable ring device) may acquire distal temperatures at the user's finger (e.g., any finger). For example, one or more temperature sensors 840 on the wearable device 804 may acquire a user's temperature from the underside of a finger or at a different location on the finger. In some implementations, the wearable device 804 may continuously acquire distal temperature (e.g., at a sampling rate). Although distal temperature measured by a wearable device 804 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the wearable device 804 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The wearable device 804 may include a PPG system 835. The PPG system 835 may include one or more optical transmitters that transmit light. The PPG system 835 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 835 may indicate the perfusion of blood in the illuminated region. For example, the PPG signal may indicate blood volume changes in the illuminated region caused by a user's pulse pressure. The processing module 830-a may sample the PPG signal and determine a user's pulse waveform based on the PPG signal. The processing module 830-a may determine a variety of physiological parameters based on the user's pulse waveform, such as a user's respiratory rate, heart rate, HRV, oxygen saturation, and other circulatory parameters.

In some implementations, the PPG system 835 may be configured as a reflective PPG system 835 where the optical receiver(s) receive transmitted light that is reflected through the region of the user's finger. In some implementations, the PPG system 835 may be configured as a transmissive PPG system 835 where the optical transmitter(s) and optical receiver(s) are arranged opposite to one another, such that light is transmitted directly through a portion of the user's finger to the optical receiver(s).

The number and ratio of transmitters and receivers included in the PPG system 835 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 835.

The PPG system 835 illustrated in FIG. 8 may include a reflective PPG system 835 in some implementations. In these implementations, the PPG system 835 may include a centrally located optical receiver (e.g., at the bottom of the wearable device 804) and two optical transmitters located on each side of the optical receiver. In this implementation, the PPG system 835 (e.g., optical receiver) may generate the PPG signal based on light received from one or both of the optical transmitters. In other implementations, other placements, combinations, and/or configurations of one or more optical transmitters and/or optical receivers are contemplated.

The processing module 830-a may control one or both of the optical transmitters to transmit light while sampling the PPG signal generated by the optical receiver. In some implementations, the processing module 830-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 835 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 830-a may store the pulse waveform in memory 815 in some implementations. The processing module 830-a may process the pulse waveform as it is generated and/or from memory 815 to determine user physiological parameters described herein.

The processing module 830-a may determine the user's heart rate based on the pulse waveform. For example, the processing module 830-a may determine heart rate (e.g., in beats per minute) based on the time between peaks in the pulse waveform. The time between peaks may be referred to as an interbeat interval (IBI). The processing module 830-a may store the determined heart rate values and IBI values in memory 815.

The processing module 830-a may determine HRV over time. For example, the processing module 830-a may determine HRV based on the variation in the IBIs. The processing module 830-a may store the HRV values over time in the memory 815. Moreover, the processing module 830-a may determine the user's respiratory rate over time. For example, the processing module 830-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 830-a may store user respiratory rate values over time in the memory 815.

The wearable device 804 may include one or more motion sensors 845, such as one or more accelerometers (e.g., 6-D accelerometers) and/or one or more gyroscopes (gyros). The motion sensors 845 may generate motion signals that indicate motion of the sensors. For example, the wearable device 804 may include one or more accelerometers that generate acceleration signals that indicate acceleration of the accelerometers. As another example, the wearable device 804 may include one or more gyro sensors that generate gyro signals that indicate angular motion (e.g., angular velocity) and/or changes in orientation. The motion sensors 845 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 830-a may sample the motion signals at a sampling rate (e.g., 50 Hz) and determine the motion of the wearable device 804 based on the sampled motion signals. For example, the processing module 830-a may sample acceleration signals to determine acceleration of the wearable device 804. As another example, the processing module 830-a may sample a gyro signal to determine angular motion. In some implementations, the processing module 830-a may store motion data in memory 815. Motion data may include sampled motion data as well as motion data that is calculated based on the sampled motion signals (e.g., acceleration and angular values).

The wearable device 804 may store a variety of data described herein. For example, the wearable device 804 may store temperature data, such as raw sampled temperature data and calculated temperature data (e.g., average temperatures). As another example, wearable device 804 may store PPG signal data, such as pulse waveforms and data calculated based on the pulse waveforms (e.g., heart rate values, IBI values, HRV values, and respiratory rate values). The wearable device 804 may also store motion data, such as sampled motion data that indicates linear and angular motion.

The wearable device 804, or other computing device, may calculate and store additional values based on the sampled/calculated physiological data. For example, the processing module 830 may calculate and store various metrics, such as sleep metrics (e.g., a Sleep Score), activity metrics, and readiness metrics. In some implementations, additional values/metrics may be referred to as “derived values.” The wearable device 804, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the wearable device 804 is oriented on the user's finger and if the wearable device 804 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 830-a may compress the data stored in memory 815. For example, the processing module 830-a may delete sampled data after making calculations based on the sampled data. As another example, the processing module 830-a may average data over longer periods of time in order to reduce the number of stored values. In a specific example, if average temperatures for a user over one minute are stored in memory 815, the processing module 830-a may calculate average temperatures over a five minute time period for storage, and then subsequently erase the one minute average temperature data. The processing module 830-a may compress data based on a variety of factors, such as the total amount of used/available memory 815 and/or an elapsed time since the wearable device 804 last transmitted the data to the user device 806.

Although a user's physiological parameters may be measured by sensors included on a wearable device 804, other devices may measure a user's physiological parameters. For example, although a user's temperature may be measured by a temperature sensor 840 included in a wearable device 804, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the wearable device 804 can make physiological measurements in a resting/sleep state in order to acquire cleaner physiological signals. In one example, the wearable device 804 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the wearable device 804 may be configured to collect, store, and/or process data, and may transfer any of the data described herein to the user device 806 for storage and/or processing. In some aspects, the user device 806 includes a wearable application 850, an operating system 885 (OS), a web browser application (e.g., web browser 880), one or more additional applications, and a GUI 875. The user device 806 may further include other modules and components, including sensors, audio devices, haptic feedback devices, and the like. The wearable application 850 may include an example of an application (e.g., “app”) that may be installed on the user device 806. The wearable application 850 may be configured to acquire data from the wearable device 804, store the acquired data, and process the acquired data as described herein. For example, the wearable application 850 may include a user interface (UI) module 855, an acquisition module 860, a processing module 830-b, a communication module 820-b, and a storage module (e.g., database 865) configured to store application data.

In some cases, the wearable device 804 and the user device 806 may be included within (or make up) the same device. For example, in some cases, the wearable device 804 may be configured to execute the wearable application 850, and may be configured to display data via the GUI 875.

The various data processing operations described herein may be performed by the wearable device 804, the user device 806, the servers 810, or any combination thereof. For example, in some cases, data collected by the wearable device 804 may be pre-processed and transmitted to the user device 806. In this example, the user device 806 may perform some data processing operations on the received data, may transmit the data to the servers 810 for data processing, or both. For instance, in some cases, the user device 806 may perform processing operations that require relatively low processing power and/or operations that require a relatively low latency, whereas the user device 806 may transmit the data to the servers 810 for processing operations that require relatively high processing power and/or operations that may allow relatively higher latency.

In some aspects, the wearable device 804 (e.g., wearable ring device), user device 806, and server 810 of the system 800 may be configured to evaluate sleep patterns for a user. In particular, the respective components of the system 800 may be used to collect data from a user via the wearable device 804, and generate one or more scores (e.g., Sleep Score, Readiness Score) for the user based on the collected data. For example, as noted previously herein, the wearable device 804 of the system 800 may be worn by a user to collect data from the user, including temperature, heart rate, HRV, and the like. Data collected by the wearable device 804 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the wearable device 804 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 800 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 700 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

It should be noted that the methods described above describe possible implementations, and that the operations and the steps may be rearranged or otherwise modified and that other implementations are possible. Furthermore, aspects from two or more of the methods may be combined.

The following provides an overview of aspects of the present disclosure:

Aspect 1: A wearable ring device, comprising: a housing; one or more light sources disposed at least partially within the housing; a layer of glass at least partially coupled to the housing and positioned to receive at least a first portion of light emitted from the one or more light sources; one or more reflective surfaces coupled to the layer of glass and positioned to reflect at least a portion of the first portion of light; one or more detectors disposed at least partially within the housing and configured to receive the portion of the first portion of light.

Aspect 2: The wearable ring device of aspect 1, further comprising: one or more additional reflective surfaces coupled to a flexible printed circuit board, wherein the one or more light sources and the one or more detectors are disposed on the flexible printed circuit board.

Aspect 3: The wearable ring device of any of aspects 1 through 2, wherein the one or more reflective surfaces are adhered to a partially-partially-domed portion of the layer of glass positioned opposite of the one or more light sources.

Aspect 4: The wearable ring device of any of aspects 1 through 3, wherein the one or more reflective surfaces are disposed along at least a portion of an inner surface of the layer of glass.

Aspect 5: The wearable ring device of any of aspects 1 through 4, wherein the one or more reflective surfaces are disposed along at least a portion of an inner surface of the layer of glass and along at least a portion of an outer surface of the layer of glass opposite of the inner surface, the one or more reflective surfaces are configured to form a light guide along the layer of glass to reflect the portion of the first portion of light through the light guide.

Aspect 6: The wearable ring device of any of aspects 1 through 5, further comprising: a microprism coupled to the layer of glass and positioned opposite of the one or more light sources, wherein the microprism is configured to couple the portion of the first portion of light into the layer of glass.

Aspect 7: The wearable ring device of any of aspects 1 through 6, further comprising: a phosphor material disposed within a partially-partially-domed portion of the layer of glass, wherein the phosphor material comprises properties that allow the portion of the first portion of light to enter the phosphor material at a first wavelength and exit the phosphor material at a second wavelength different than the first wavelength.

Aspect 8: The wearable ring device of any of aspects 1 through 7, wherein the one or more light sources comprise a light emission pattern, and the one or more reflective surfaces are configured to modify a set of characteristics of the light emission pattern towards the one or more detectors.

Aspect 9: The wearable ring device of any of aspects 1 through 8, wherein the set of characteristics of the light emission pattern comprises a light emission direction, a light emission tilt angle, a light emission size, a light emission shape, or a combination thereof.

Aspect 10: The wearable ring device of any of aspects 1 through 9, wherein the one or more detectors comprise a field of view, and wherein the one or more reflective surfaces are configured to adjust an overlapping portion of the field of view and the light emission pattern.

Aspect 11: The wearable ring device of any of aspects 1 through 10, wherein the layer of glass comprises one or more total internal reflection surfaces, one or more micro-optical structures, one or more uneven surfaces, or a combination thereof.

Aspect 12: The wearable ring device of any of aspects 1 through 11, wherein the one or more reflective surfaces comprise a reflective material, an opaque material, a reflective coating, a diffuse white coating, or a combination thereof.

Aspect 13: The wearable ring device of any of aspects 1 through 12, wherein the one or more light sources comprise one or more green light-emitting diodes, one or more red light-emitting diodes, one or more infrared light sources, a blue laser diode, or any combination thereof.

Aspect 14: The wearable ring device of any of aspects 1 through 13, wherein the housing comprise a ring-shaped housing.

Aspect 15: The wearable ring device of any of aspects 1 through 14, further comprising: a titanium oxide material disposed within the layer of glass, wherein the titanium oxide material comprises properties that prevent the first portion of light from entering the layer of glass.

Aspect 16: The wearable ring device of any of aspects 1 through 15, further comprising: one or more metallic wires disposed within a portion of the layer of glass, wherein the one or more metallic wires are configured to prevent the first portion of light from entering the portion of the layer of glass.

Aspect 17: A wearable ring device, comprising: an inner ring-shaped housing and an outer ring-shaped housing; one or more light sources disposed on a flexible printed circuit board positioned between the inner ring-shaped housing and the outer ring-shaped housing; a layer of partially-domed glass at least partially coupled with the inner ring-shaped housing and positioned to absorb at least a first portion of light emitted from the one or more light sources; and one or more reflective coatings adhered to the layer of partially-domed glass and positioned to reflect at least a portion of the first portion of light; one or more detectors disposed on the flexible printed circuit board positioned between the inner ring-shaped housing and the outer ring-shaped housing, wherein the one or more detectors are configured to receive the portion of the first portion of light.

Aspect 18: The wearable ring device of aspect 17, further comprising: one or more additional reflective coatings adhered to the flexible printed circuit board and positioned adjacent to the one or more light sources, wherein the one or more additional reflective coatings are configured to reflect a subset of the portion of the first portion of light into the layer of partially-domed glass.

Aspect 19: The wearable ring device of any of aspects 17 through 18, wherein the one or more reflective coatings are adhered along an inner surface of the layer of partially-domed glass and an outer surface of the layer of partially-domed glass to form a light guide that reflects the portion of the first portion of light between the inner surface and outer surface and along the layer of partially-domed glass.

Aspect 20: The wearable ring device of any of aspects 17 through 19, further comprising: a phosphor material disposed within the layer of partially-domed glass, wherein the phosphor material comprises properties that allow the portion of the first portion of light to enter the phosphor material at a first wavelength corresponding to blue light and exit the phosphor material at a second wavelength corresponding to yellow light, green light, or red light.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable ring device, comprising:
a housing;
one or more light sources disposed at least partially within the housing;
a layer of glass at least partially coupled to the housing and positioned to receive at least a first portion of light emitted from the one or more light sources;
one or more reflective surfaces coupled to the layer of glass and positioned to reflect at least a portion of the first portion of light;
one or more detectors disposed at least partially within the housing and configured to receive the portion of the first portion of light.

2. The wearable ring device of claim 1, further comprising:
one or more additional reflective surfaces coupled to a flexible printed circuit board, wherein the one or more light sources and the one or more detectors are disposed on the flexible printed circuit board.

3. The wearable ring device of claim 1, wherein the one or more reflective surfaces are adhered to a partially-partially-domed portion of the layer of glass positioned opposite of the one or more light sources.

4. The wearable ring device of claim 1, wherein the one or more reflective surfaces are disposed along at least a portion of an inner surface of the layer of glass.

5. The wearable ring device of claim 1, wherein
the one or more reflective surfaces are disposed along at least a portion of an inner surface of the layer of glass and along at least a portion of an outer surface of the layer of glass opposite of the inner surface,
the one or more reflective surfaces are configured to form a light guide along the layer of glass to reflect the portion of the first portion of light through the light guide.

6. The wearable ring device of claim 1, further comprising:
a microprism coupled to the layer of glass and positioned opposite of the one or more light sources, wherein the microprism is configured to couple the portion of the first portion of light into the layer of glass.

7. The wearable ring device of claim 1, further comprising:
a phosphor material disposed within a partially-partially-domed portion of the layer of glass, wherein the phosphor material comprises properties that allow the portion of the first portion of light to enter the phosphor material at a first wavelength and exit the phosphor material at a second wavelength different than the first wavelength.

8. The wearable ring device of claim 1, wherein
the one or more light sources comprise a light emission pattern, and
the one or more reflective surfaces are configured to modify a set of characteristics of the light emission pattern towards the one or more detectors.

9. The wearable ring device of claim 8, wherein the set of characteristics of the light emission pattern comprises a light emission direction, a light emission tilt angle, a light emission size, a light emission shape, or a combination thereof.

10. The wearable ring device of claim 9, wherein the one or more detectors comprise a field of view, and wherein the one or more reflective surfaces are configured to adjust an overlapping portion of the field of view and the light emission pattern.

11. The wearable ring device of claim 1, wherein the layer of glass comprises one or more total internal reflection surfaces, one or more micro-optical structures, one or more uneven surfaces, or a combination thereof.

12. The wearable ring device of claim 1, wherein the one or more reflective surfaces comprise a reflective material, an opaque material, a reflective coating, a diffuse white coating, or a combination thereof.

13. The wearable ring device of claim 1, wherein the one or more light sources comprise one or more green light-emitting diodes, one or more red light-emitting diodes, one or more infrared light sources, a blue laser diode, or any combination thereof.

14. The wearable ring device of claim 1, wherein the housing comprise a ring-shaped housing.

15. The wearable ring device of claim 1, further comprising:
a titanium oxide material disposed within the layer of glass, wherein the titanium oxide material comprises properties that prevent the first portion of light from entering the layer of glass.

16. The wearable ring device of claim 1, further comprising:
one or more metallic wires disposed within a portion of the layer of glass, wherein the one or more metallic wires are configured to prevent the first portion of light from entering the portion of the layer of glass.

17. A wearable ring device, comprising:
an inner ring-shaped housing and an outer ring-shaped housing;
one or more light sources disposed on a flexible printed circuit board positioned between the inner ring-shaped housing and the outer ring-shaped housing;
a layer of partially-domed glass at least partially coupled with the inner ring-shaped housing and positioned to absorb at least a first portion of light emitted from the one or more light sources; and
one or more reflective coatings adhered to the layer of partially-domed glass and positioned to reflect at least a portion of the first portion of light;
one or more detectors disposed on the flexible printed circuit board positioned between the inner ring-shaped housing and the outer ring-shaped housing, wherein the one or more detectors are configured to receive the portion of the first portion of light.

18. The wearable ring device of claim 17, further comprising:
one or more additional reflective coatings adhered to the flexible printed circuit board and positioned adjacent to the one or more light sources, wherein the one or more additional reflective coatings are configured to reflect a subset of the portion of the first portion of light into the layer of partially-domed glass.

19. The wearable ring device of claim 17, wherein the one or more reflective coatings are adhered along an inner surface of the layer of partially-domed glass and an outer surface of the layer of partially-domed glass to form a light guide that reflects the portion of the first portion of light between the inner surface and outer surface and along the layer of partially-domed glass.

20. The wearable ring device of claim 17, further comprising:
a phosphor material disposed within the layer of partially-domed glass, wherein the phosphor material comprises properties that allow the portion of the first portion of light to enter the phosphor material at a first wavelength corresponding to blue light and exit the phosphor material at a second wavelength corresponding to yellow light, green light, or red light.
